(19) United States
(12) Patent Application Publication
(10) Pub. No.: US 2010/0260835 A1
(43) Pub. Date: Oct. 14, 2010

(54) PHARMACEUTICAL COMPOSITION

(76) Inventors: Derek Alfred Wheeler, Surrey (GB); David Fraser Steele, Surrey (GB)

Correspondence Address: MANNAVA & KANG, P.C., 11240 WAPLES MILL ROAD, Suite 300, FAIRFAX, VA, 22030, US

(21) Appl. No.: 12/666,505

(22) PCT Filed: Jun. 26, 2008
(86) PCT No.: PCT/GB08/02212

§ 371 Date: May 24, 2010

(30) Foreign Application Priority Data
Jun. 26, 2007 (GB) 0712389.6

(51) Int. Cl. A61K 9/10; A61K 38/13; A61K 31/137; A61K 9/48; A61P 33/06; A61P 17/00; A61P 27/14; A61P 25/22; A61P 9/06; A61P 11/06; A61P 3/10; A61P 25/24; A61P 25/28; A61P 25/30; A61P 9/04; A61P 25/16; A61P 35/00

(52) U.S. Cl. 424/455; 514/20.5; 424/484; 514/653; 424/485; 424/488; 424/486

(57) ABSTRACT

A pharmaceutical composition comprising a moulded body of a polymer matrix, said polymer matrix comprising at least one polyaphron dispersion and at least one pharmaceutically active agent dispersed therein.

The present invention relates to a pharmaceutical composition. In particular, the present invention relates to a pharmaceutical composition comprising at least one polyaphron dispersion and at least one pharmaceutically active agent.

The oral delivery of poorly soluble drugs is known to be unsatisfactory when the drug is administered as a simple solution in a lipophilic medium. Attempts to address this problem have led to the development of lipid-based delivery vehicles such as those reviewed in—for example, Humberstone & Charman (1997) Advanced Drug Delivery Review v.25, 103-128 and O'Driscoll (2002) European Journal of Pharmaceutical Science v.15, 405-415.

More effective delivery of such drugs has been achieved by the use of self-emulsifying drug delivery systems (SEDDS). SEDDS comprise a mixture of an oil and a surfactant that spontaneously forms an oil-in-water emulsion when diluted with water. The solubility of the drug is typically enhanced by the presence of the surfactant—which is usually present in concentrations as high as or greater than 30% by weight. The main disadvantage of SEDDS relates to the presence of the large amounts of surfactant, which, apart from potentially having a harmful effect on the intestinal wall, adds to the cost and complexity of the formulation. Examples of such compositions are disclosed in U.S. Pat. Nos. 6,436,430 and 6,284,268.

Recently the present inventors have disclosed the use of biliquid foams in drug delivery systems. In particular, International patent application WO 2005/011628 describes an oral drug delivery system comprising a biliquid foam comprising from 1 to 20% by weight of a continuous hydrophilic phase, from 70 to 98% by weight of a pharmaceutically acceptable oil, said pharmaceutically acceptable oil having dissolved or dispersed therein a poorly water soluble drug in amount of from 0.1 to 20% by weight, and the biliquid foam including therein from 0.5 to 5% by weight of a surfactant, all percentages being based upon the total weight of the formulation. Unlike the SEDDS mentioned above, the oral drug delivery systems described in WO 2005/011628 do not require high levels of surfactant. WO 2005/011628 also discloses capsules filled with biliquid foam, for example, hard or soft coated gelatine capsules.

A disadvantage of the drug delivery systems comprising capsules filled with biliquid foam is that the walls of the capsule may absorb significant amounts of water from the biliquid foam contents giving rise to instability of the biliquid foam. This problem has been found to be particularly problematic when there are high water levels in the biliquid foams. WO 2005/011628 mitigates this problem by limiting the biliquid foam to having a continuous hydrophilic phase from 1 to 20% by weight of the total composition. However, the need to maintain compatibility between the capsule contents and the materials of the wall of the capsule has, until now, limited the range of formulation options available.

A further disadvantage of hard or soft coated capsules is that once the formulation has reached the gastrointestinal tract, there is a sudden release of the drug from the drug carrying system when the capsule coating dissolves and/or disintegrates. This is not always desirable, for example, if the absorption of the drug through the lining of the gut is slow. In this case a more extended release profile may be preferable so as not to lose a significant proportion of the released drug by passage further through the tract before absorption can take place.

US patent application US 2005/0238676 A1 describes a discrete, free-flowing powder which comprises particles in which a biliquid foam is trapped within a matrix of polymer material. Such a powder is preferably produced by a spray dry or freeze dry method as the result of which the final form of the powder contains less than 1% of water. The powder as described in US 2005/0238676 A1 has the disadvantage that its consistency, hardness or softness cannot be varied to produce a pharmaceutical composition which may be applied directly to and bioadhere to, for example, human or animal skin or to a mucosal surface of an animal or human to deliver an active agent.

A further limitation of the powder of US patent application US 2005/0238676 A1 is that the powder is incapable of carrying more than 50% by weight, and typically not more than 40% by weight of oil droplets based on the total weight of the powder, limiting its ability to deliver physiologically active concentrations of oil soluble drugs. The polyaphron dispersion (biliquid foam) content of the present invention can surprisingly be as high as 70% by weight based on the total weight of the pharmaceutical composition, of which the oil content can be as high as 95% by weight.

There is a need to formulate an improved drug delivery system which addresses at least some of the problems of the prior art.

The present inventors have surprisingly found that at least some of the problems of instability can be addressed by formulating a composition comprising a moulded body of a polymer matrix, for example formed by gelatine and/or other suitable polymers, at least one polyaphron dispersion and at least one pharmaceutically active agent into a single homogenous mass capable of being moulded, preferably by sol-gel transition, into a desired shape. This moulded body may then further be encapsulated with hard or soft gelatine, or coated with a suitable coating for protection or identification, if desired. The pharmaceutical composition of the present invention provides for the gradual release of the pharmaceutically active agent(s) over an extended period of time as the polymer dissolves and/or disintegrates.

Accordingly, the present invention provides a pharmaceutical composition comprising a moulded body of a polymer matrix, said polymer matrix comprising at least one polyaphron dispersion and at least one pharmaceutically active agent dispersed therein.

According to another aspect of the present invention there is provided the use of the pharmaceutical composition as described above for the manufacture of a medicament for treating allergic reactions, alopecia, anaemia, angina, anxiety disorders, arrhythmia, arthritis, asthma, bites and stings, bone diseases, cancer, coughs, dementia, depression, diabetes, drug addiction, eczema, elevated cholesterol, epilepsy, extrapyramidal disorders, fever and hyperthermia, gastrointestinal disorders, gout, heart failure, hormonal disorders, hyperlipidaemia, hypertension, hypochondria, hypothalamic and pituitary disorders, infections (bacterial, ectoparastic, fungal, helminthic, protozoal, viral), inflammation, insomnia and parasomnias, protozoal infections, kidney disorders, labour induction, liver disorders, local and systemic pain, malaria, migraine, muscular dystrophies, myasthemia, nasal congestion, nutritional deficiencies, parathyroid disorders, Parkinson's disease, poisoning, premature labour, provide sedation, psychoses, respiratory disorders, rheumatism, salt retention, shock, skin disorders (acne, dermatitis, keratinisation disorders, psoriasis, ulcers, warts), thromboembolic disorders, tapeworm infection, thyroid imbalance, tuberculosis, urological disorders, water retention, local or systemic pain or xenograft rejection.

According to another aspect of the present invention there is provided a method of making the pharmaceutical composition as defined above comprising mixing a polyaphron dispersion with a polymer to form a polymer matrix having a polyaphron dispersion and at least one pharmaceutically active agent dispersed therein, and moulding the mixture into a desired shape.

By polyaphron dispersion as used herein is meant a particular kind of hydrophilic liquid-in-hydrophobic liquid or hydrophobic liquid-in-hydrophilic liquid dispersion comprising (a) a hydrophilic liquid miscible phase, (b) a second hydrophobic phase being immiscible or substantially immiscible with the first phase and (c) one or more surfactants, wherein the dispersed or discontinuous phase is in the form of small (e.g. micron to sub-micron diameter, but more usually at least 1 micron diameter) droplets, and the whole having the following characteristics, which distinguish polyaphron dispersions from conventional or common emulsions and other dispersion types:
1. They are capable of existing in a stable form wherein the volume fraction of the dispersed phase (φᵢₚ) is greater than 0.7 and can be as high as 0.97. (φᵢₚ is the volume ratio of discontinuous to continuous phase expressed as a fraction).
2. The microscopic appearance of polyaphron dispersions where φᵢₚ is greater than 0.7 is that of an aggregate of individual droplets, pushed closely together into polyhedral shapes, resembling the appearance of a gas foam. In this form, the dispersion has gel-like properties and is referred to as a Gel Polyaphron Dispersion (GPD).
3. Stable polyaphron dispersions can be formed with a surfactant concentration less than 3% and more typically less than 2% by weight of the total composition.
4. Gel Polyaphron Dispersions (as described in 2 above) can be diluted to any extent by the addition of more continuous phase without the addition of more surfactant, when the gel-like properties disappear. Once φᵢₚ has been reduced to below 0.7, the individual droplets of internal phase become separated to take the form of spherical droplets, which remain stable and intact but which may nevertheless join together in loose associations and float to the top or sink to the bottom of the diluted dispersion (depending on the relative densities of the two phases). In this diluted form each droplet is referred to as a Colloidal Liquid Aphron (CLA). Simple shaking of the diluted dispersion instantly causes a homogeneous, stable dispersion of Colloidal Liquid Aphrons to re-form.
   Each of the above characteristics and a combination of them clearly differentiate the polyaphron dispersions of the present invention from conventional emulsions and other dispersion types which do not have all of those characteristics. Polyaphron dispersions are disclosed in the following literature references by Sebba: “Biliquid Foams”, J. Colloid and Interface Science, 40 (1972) 468-474 and “The Behaviour of Minute Oil Droplets Encapsulated in a Water Film”, Colloid Polymer Sciences, 257 (1979) 392-396, Hicks “Investigating the Generation, Characterisation, and Structure of Biliquid Foams”, PhD Thesis, University of Bristol, 2005, Crutchley “The Encapsulation of Oils and Oil Soluble Substances Within Polymer Films”, PhD Thesis, The University of Leeds, 2006 and Lye and Stuckey, Colloid and Surfaces, 131 (1998) 119-136. Aphrons are also disclosed in U.S. Pat. No. 4,486,333 and WO 97/32559.

Polyaphron dispersions are sometimes referred to as ‘Biliquid Foams’, ‘High Internal Phase Emulsions (HIPEs)’, ‘High Internal Phase Ratio Emulsions (HIPREs)’ and ‘Gel Emulsions’. All such descriptions that refer to dispersions having the characteristics described above are polyaphron dispersions as used in the present invention.

By ‘mouldable by sol-gel transition’ as used herein is meant the process whereby a liquid (sol) is poured into the cavity of a mould after which it is converted, without the use of external mechanical forces (for example without the use of additional pressure), into a body having a gel-like consistency which, when removed from the mould, retains the essential shape of the mould cavity. By ‘gel-like consistency’ is meant a body having a constancy ranging from a highly deformable jelly to a stiff non-deformable structure.

Each aspect as defined herein may be combined with any other aspect or aspects unless clearly indicated to the contrary. In particular any feature indicated as being preferred or advantageous may be combined with any other feature or features indicated as being preferred or advantageous.

The polymer used in the present invention is a water-dispersible and/or a water-soluble polymer and is capable of forming a moulded body having at least one polyaphron dispersion and at least one pharmaceutically active agent dispersed therein. The pharmaceutical composition may be designed, for example, such that when it is placed in contact with water, or an aqueous environment (such as saliva, gastric juices, or plasma from open wounds) the polymer disintegrates and/or dissolves, releasing the aphrons and the pharmaceutically active agent. The polymer may be chosen such that although at room temperature it is not water soluble or water dispersible, under the conditions which prevail in the human body, the polymer is water soluble and/or water dispersible.

When released, the liberated aphrons are water-dispersible and have a high surface area to volume ratio thus providing a greater probability of absorption of the dissolved active agents than for many other forms of drug delivery, especially for oil soluble actives.

Suitable polymers for use in the present invention are preferably capable of undergoing a sol-gel transition by reason of temperature change (for example, gelatine) or by reason of crosslinking (for example alginate salts) and include, for example, gelatine, agar, carrageenan, alginates, other water dispersible or water soluble mouldable polymers known in the art. Mixtures of the above may also be used in the present invention.

Preferred polymers include gelatine and careeganan gum. Most preferably, the polymer is gelatine.

The pharmaceutical composition preferably comprises from 10 to 50% by weight, more preferably from 10 to 40% by weight, more preferably still from 10 to 20% by weight of polymer, all percentages being based on the total weight of the pharmaceutical composition.

By careful selection of the polymer(s) the rate of dispersion and/or dissolution of the polymer matrix may be controlled. This also allows the rate of dispersion and/or dissolution of the pharmaceutically active agent(s) to be varied. Other means of controlling the rate of dispersion and/or dissolution of the polymer matrix includes cross-linking of the polymer. Methods of cross-linking polymers are well known in the art. In addition, or instead of cross-linking the polymer in order to effect the rate of dispersion and/or dissolution of the polymer matrix, a polymer which is not capable of a sol-gel transition may be included in the polymer matrix. The inclusion of such a polymer as a component of the matrix may modify the dispersion and/or dissolution characteristics.

Preferably in order to make the moulded body of the matrix of the present invention, the polymer is mixed with a solvent. The solvent is chosen such that the polymer may be dissolved and/or dispersed therein. It may be necessary to heat the solvent to in order to dissolve/disperse suitable amounts of the polymer therein. Suitable solvents include, but are not limited to, aliphatic alcohols, polyethylene glycol, propylene glycol, glycerol, and mixtures thereof. Most preferably the solvent is water. Suitable aliphatic alcohols include, for example, ethanol, propanol, and isopropanol.

Preferably the pharmaceutical composition of the present invention comprises at least 10% by weight of solvent based on the total weight of the composition. More preferably the pharmaceutical composition comprises at least 20% by weight, at least 40% by weight, at least 60% by weight or at least 65% by weight of solvent based on the total weight of the composition. Preferably the solvent is water.

Advantageously the composition of the present invention may comprise high levels of solvent in the composition (i.e. greater than 10%, 20%, 20%, 50% 60%, or 65% by weight of solvent based on the total weight of the composition) whilst still enabling the composition to be moulded into a desired shape.

A plasticizer or a mixture thereof may be used in the present invention to make the pharmaceutical composition more elastic and pliable. Plasticizers may be selected from, for example, the group consisting of polyalcohol organic acids, hydroxyl acids, amines, acid amines, sulphoxides and pyrrolidones. In a preferred embodiment of the present invention the polyalcohol organic acids are selected from the group of sorbitol, mannitol, glycerol, xylitol, maltitol, Maltisorb®, propylene glycol, polyethylene glycol, lactitol, trehalose, sorbitan esters and sorbitol anhydride and mixtures thereof.

By careful choice of identity and concentration of polymers, products made using the present invention can be made to have a bioadhesive surface. Such a surface would be useful when, for example, the product is in the form of a patch for use on wounds. As used herein, the term “bioadhesive surface” refers to a property of the surface of the product that enables it to adhere to a biological surface, such as a mucous membrane or skin, preferably for an extended period of time.

Preferably the moulded body of the polymer matrix comprises cross-linked polymers.

The pharmaceutical composition preferably comprises from 0 to 10.0% by weight, more preferably from 0.5 to 5.0% by weight, more preferably still from 1.0 to 2.0% by weight of a plasticizer, all percentages being based on the total weight of the pharmaceutical composition.

It will be understood that the amount and choice of the plasticizer will help to determine the hardness of the final product. It may also effect the disintegration and/or dissolution of the moulded body, as well as its physical and chemical stability.

It will be understood that the consistency of the moulded body may be varied from a gel-like consistency to a solid consistency by careful choice of ingredients of the pharmaceutical composition. In one embodiment of the present invention the moulded polymer matrix is in the form of a semi-solid colloidal gel.

In a preferred embodiment, the polymer has a controllable and predictable sol-gel transition, and once in the form of a sol can be poured into a mould and is capable of being moulded into the desired shape. The sol-gel transition may be made to occur, for example, due to a change in temperature.

In another embodiment, the polymer is initially in a fluid or semi-fluid state in such a form as to be capable of being poured or injected into a suitable mould. Transition to a gel form may then be promoted by the addition or controlled release of a cross-linking agent such as a metal ion.

In the method of making the pharmaceutical composition as defined herein appropriate steps may be taken in order to avoid or reduce any degradation or loss of the pharmaceutical agent. One skilled in the art will be aware of appropriate steps that may be taken. These may include control of temperature, atmosphere, contaminants and light. For example the method of making the pharmaceutical composition may require an inert flowing gas with appropriate filtration and in a darkened environment in order to avoid decomposition or contamination of the pharmaceutical agent. Other specific factors may also need to be addressed which would be obvious to one skilled in the art.

The polymer may be chosen such that it is sensitive to acidity or alkalinity so that the release of the entrapped pharmaceutically active agent(s) may be determined by a change of pH or by the presence of another chemical species.

In a preferred embodiment the moulded body is in the form of a capsule, tablet, suppository, pessary, depot, lozenge or film. It will be understood that the pharmaceutical composition may be moulded into other suitable shapes, for example, for masticating, swallowing or dissolving in the mouth, or for applying to an open wound or inserting into the rectum or vagina, into the ear or used as an eye patch or a wound dressing as required. It will be further understood that the moulded body may have a symmetrical or asymmetrical shape.

It will be understood that in some circumstances care may need to be taken to prevent damage to vulnerable dissolved or dispersed active molecules for example by suitable choice of temperature and/or environment (for example, use of an inert atmosphere) during the moulding process.

One advantage of the present invention is that the mouldable bodies described above may be designed to dissolve slowly or quickly (as desired) to efficiently and effectively deliver the pharmaceutically active agent(s) to a target site.

A further advantage of the present invention is that, wherein at least one pharmaceutically active agent is an oil-soluble and/or oil-dispersible drug, it may be initially dissolved and/or dispersed in polyaphron dispersions having a very low water content. For example polyaphron dispersions having from 5-10% by volume of water or a continuous phase are easily attainable. For this reason, the concentration of the drug may be much higher than if, for example, a conventional emulsion (typically having a water or continuous phase content of 65% or greater) is used in place of the polyaphron dispersion.

A further advantage of the present invention is that more than one polyaphron dispersion may be present in the pharmaceutical composition. Each polyaphron dispersion may comprise one or more pharmaceutically active agents, which may be present in the continuous or discontinuous phase. This allows a pharmaceutical composition to be provided which may encompass pharmaceutically active agents which are incompatible with one another using traditional delivery methods without detrimental stability issues. In other prior art products, it may be impossible to find a single oil phase in which the combination of oil soluble drugs are both sufficiently soluble to provide a physiologically acceptable dose. In the present invention, these problems can be at least partially overcome by the use of more than one polyaphron dispersion, each of which may comprise a different oil comprising different pharmaceutically active agents.

A yet further advantage of the present invention is that, wherein at least one pharmaceutically active agent is oil-soluble, the oil-soluble pharmaceutically active agent may not have to be subjected to the high manufacturing temperatures usually required by stable conventional emulsions (70-90° C.) and is therefore less likely to be damaged by heat.

The pharmaceutical composition of the present invention may comprise a water-dispersible and/or water-soluble outer coating. The outer coating may comprise, for example, hard or soft gelatine, shellac, methacrylic acid copolymers, cellulose acetate, hydroxypropylmethyl cellulose phthalate, polyvinylacetate phthalate, sugar, or sugar alcohols, such as sorbitol or xylitol. Such a coating may provide rigidity and control the site of disintegration and/or dissolution of the pharmaceutical composition in, for example, the gut.

In a preferred embodiment the moulded body is a film. More preferably still, the moulded body is a film having a size such that it can be placed in the oral cavity. Preferably, the film is designed to exist as a free moulded body, i.e. not attached to a substrate.

Wherein the film is designed to exist as a free moulded body, preferably the thickness of the film is from 0.1 to 5.0 mm, more preferably from 0.2 to 1.0 mm and more preferably still from 0.5 to 0.8 mm.

Advantageously, the moulded composition may consist of two or more layers, each layer may have a different level of active agent and/or it may have a different solubility, for example in the environment of the gastro-intestinal (GI) tract.

In one embodiment of the present invention, the pharmaceutical composition may comprise a moulded body as described herein which forms an outer layer which may be, for example, soluble in the acid environment of the stomach and a further moulded body as described herein which forms an inner layer which may be, for example, soluble only at the pH of the lower intestine. In this way, one or more pharmaceutically active agents may be differentially released.

In another embodiment, each layer may contain active agents that are mutually incompatible when mixed and stored in a single conventional composition.

In yet another embodiment, multiple layers containing active agents may be separated by interleaved layers of water-insoluble polymers or polymers having solubilities that vary with pH.

Advantageously, the composition of the present invention may be multi-layered whereby each additional layer concentrically encloses the core or layer beneath it.

In another embodiment, the layers may be arranged sequentially along the length of the moulded composition such that each end is composed of a different layer.

Unlike, for example, the surface coating films disclosed in WO 99/05229, in which the aphrons are designed to “dry out” such that the weight of the hydrophilic phase of the aphrons in the film is significantly less than the weight of hydrophilic phase in the aphrons prior to film forming, the present invention is designed such that the amount of hydrophilic phase remains substantially the same in the moulded body as in the aphron dispersions prior to forming the composition of the present invention.

It has surprisingly been found that the amount of polyaphron dispersions in the polymer matrix may be as high as 70% by weight of the total composition. Preferably, the amount of polyaphron dispersions is at least 20%, more preferably at least 30%, more preferably still at least 40% by weight of the total composition.

The pharmaceutical composition preferably comprises from 1 to 70% by weight, more preferably from 25 to 70% by weight, or from 25 to 60% by weight, more preferably still from 45 to 70% by weight, or from 45 to 60% by weight of polyaphron dispersions, all percentages being based on the total weight of the pharmaceutical composition.

The polyaphron dispersions comprise a continuous phase, a discontinuous phase and a surfactant. Preferably, the discontinuous phase comprises a pharmaceutically acceptable oil phase, and the continuous phase is hydrophilic.

Examples of oils which may be used in the present invention include almond oil, babassu oil, blackcurrant seed oil, borage oil, canola oil, castor oil, coconut oil, cod liver oil, corn oil, cottonseed oil, evening primrose oil, fish oil, grapeseed oil, mustard seed oil, oat oil, olive oil, palm kernel oil, palm oil, peanut oil, rapeseed oil, safflower oil, sesame oil, squalene, squalane, soybean oil, sunflower oil, walnut oil, wheat germ oil, hydrogenated castor oil, hydrogenated coconut oil, hydrogenated cottonseed oil, hydrogenated palm oil, hydrogenated soybean oil, partially hydrogenated soybean oil, hydrogenated vegetable oil, modified triglycerides, caprylic/capric glycerides, fractionated triglycerides, glyceryl tricaprate, glyceryl tricaproate, glyceryl tricaprylate, glyceryl tricaprylate/caprate, glyceryl tricaprylate/caprate/laurate, glyceryl tricaprylate/caprate/linoleate, glyceryl tricaprylate/caprate/stearate, glyceryl trilaurate, glyceryl trilinoleate, glyceryl trilinolenate, glyceryl trioleate, glyceryl triundecanoate, linoleic glycerides, saturated polyglycolized glycerides, synthetic medium chain triglyceride containing primarily C₈-C₁₂ fatty acid chains, medium chain triglycerides, long chain triglycerides, modified triglycerides, fractionated triglycerides, and mixtures thereof.

Examples of mono and diglycerides which may be used in the present invention include propylene glycol mono and diesters having from 15 to 40 carbon atoms, including hydrolysed coconut oils (e.g. Capmul MCM), hydrolysed corn oil (e.g. Maisine 35-1).

The monoglycerides and diglycerides are mono- or di-saturated fatty acid esters of glycerol having a carbon chain length of eight to sixteen units.

Essential oils may also be used in the present invention.

Preferably the pharmaceutical composition comprises from 1 to 70% by weight, more preferably from 25 to 70% by weight, more preferably still from 45 to 70% by weight of discontinuous phase of the polyaphron dispersion, all percentages being based on the total weight of the pharmaceutical composition. Preferably the discontinuous phase is a pharmaceutically acceptable oil.

Preferably the pharmaceutical composition comprises at least 55% by weight of pharmaceutically acceptable oil, more preferably at least 60% by weight, all percentages being based on the total weight of the pharmaceutical composition.

Preferably, the average diameter of the discontinuous phases in the polyaphron dispersions are from 0.001 to 0.1 mm, more preferably from 0.002 to 0.03 mm, more preferably still from 0.003 to 0.02 mm.

The continuous hydrophilic phase of the aphrons may comprise water. The continuous hydrophilic phase may additionally comprise a co-solvent such as an aliphatic alcohol, polyethylene glycol, propylene glycol or glycerol, or mixtures thereof.

Alternatively, the hydrophilic phase may be non-aqueous and may be, for example, an aliphatic alcohol, polyethylene glycol, propylene glycol or glycerol, or mixtures thereof.

The pharmaceutical composition may comprise at least 20% by weight of continuous phase, at least 30% by weight of continuous phase, or at least 40% by weight of continuous phase based on the total weight of the pharmaceutical composition. Preferably the continuous phase is hydrophilic.

The surfactant used in the present invention may be incorporated into either or both phases of the polyaphron dispersions. The surfactant used in the present invention is preferably an alkyl polyglycol ether, an alkyl polyglycol ester, an ethoxylated alcohol, a polyoxyethylene sorbitan fatty acid ester, a polyoxyethylene fatty acid ester, an ionic or non-ionic surfactant, a hydrogenated castor oil/polyoxyethylene glycol adducts containing from 25 to 60 ethoxy groups a castor oil/polyoxyethylene glycol adduct containing from 25 to 45 ethoxy groups, a sorbitan fatty acid ester (for example Span 20 or Span 80), a block copolymer of ethylene oxide and propylene oxide (for example Pluronic L121 or Pluronic F68), or a mixture thereof.

Preferably the pharmaceutical composition comprises from 0.05 to 5% by weight, more preferably from 0.1 to 1.5% by weight, more preferably still from 0.2 to 1.0% by weight of a surfactant, all percentages being based on the total weight of the pharmaceutical composition.

Preferably the polyaphron dispersions are substantially evenly distributed throughout the moulded body of the polymer matrix.

Preferably the polyaphron dispersions are directly dispersed within the polymer which is moulded to form the pharmaceutical composition of the present invention.

Suitable pharmaceutically active agents may be selected from an analgesic or anti-inflammatory agent, an anthelmintic, an anti-arrhythmic agent, an anti-coagulant, an anti-depressant, an anti-diabetic, an anti-epileptic, an anti-fungal agent, an anti-gout agent, an anti-hypertension agent, an anti-malarial, an anti-migraine agent, an anti-muscarinic agent, an anti-neoplastic agent, an anti-protozoal agent, an anti-thyroid agent, an anxiolytic, sedative, hypnotic or neuroleptic agent, a corticosteroid, a diuretic, an anti-Parkinsonian agent, a gastro-intestinal agent, a histamine H1-receptor antagonist, a lipid regulating agent, an anti-anginal agent, a thyroid agent, a nutritional agent, an antipyretic agent, an antibacterial agent, an immunosuppressant, an antiviral agent, hypothalmic or a pituitary hormone, a sex hormone, a prostaglandin, a vaccine, a cough suppressant, a local anaesthetic, an immuno-globulin an antisera, an opioid analgesic, a stimulant, a viral vector for gene therapy, a vaccine or a therapeutic mixture thereof.

Preferably the pharmaceutical composition comprises from 0.0001 to 40% by weight, more preferably from 0.1 to 10% by weight, more preferably still from 0.1 to 2.0% by weight of at least one pharmaceutically active agent, all percentages being based on the total weight of the pharmaceutical composition.

At least one pharmaceutically active agent may be trapped in the polymer matrix of the pharmaceutical composition of the present invention. At least one pharmaceutically active agent may be at least-partially dispersed and/or dissolved in the polyaphron dispersion. In particular, at least one pharmaceutically active agent may be present in the discontinuous and/or continuous phase of the polyaphron dispersion.

In one embodiment of the present invention at least one pharmaceutically active agent is dispersed and/or dissolved in the polymer matrix and at least one pharmaceutically active agent is dispersed and/or dissolved in the polyaphron dispersion.

More than one pharmaceutically active agent may be present in the pharmaceutical composition of the present invention. The pharmaceutical composition may comprise more than one polyaphron dispersion, and each polyaphron dispersion may comprise a different pharmaceutically active agent. This may be advantageous in order to avoid compatibility issues between the different pharmaceutically active agents.

Wherein the pharmaceutically active agent is a “poorly water soluble drug”, preferably the drug is dissolved in the polyaphron dispersion. As used herein the term poorly water soluble is meant a drug which will dissolve in water in an amount of less than 1% by weight. In this embodiment it may be advantageous to use a co-emulsifier in the formation of the polyaphron dispersion in an amount sufficient to complete the solubilization of the poorly water-soluble drug. A suitable co-emulsifier is a phosphoglyceride, a phospholipid, for example lecithin, or a free fatty acid that is liquid at room temperature, for example iso-stearic acid, oleic acid, linoelic acid or linolenic acid.

Wherein the pharmaceutically active agent is a “poorly water soluble drug”, it may include the following:

Analgesics and anti-inflammatory agents: aceclofenac, aloxiprin, auranofin, azapropazone, benorylate, capsaicin, celecoxib, diflunisal, etodolac, fenbufen, fenoprofen calcium, flurbiprofen, ibuprofen, indomethacin, ketoprofen, meclofamate, mefenamic acid, meloxicam, nabumetone, naproxen, nimesulide, oxyphenbutazone, phenylbutazone, piroxicam, refocoxib, sulindac, suxibuzone, tolmetin, zileuton.

Anthelmintics: albendazole, bephenium hydroxynaphthoate, dichlorophen, ivermectin, mebendazole, oxfendazole, oxantel embonate, praziquantel, pyrantel embonate, thiabendazole.

Anti-arrhythmic agents: amiodarone HCl, disopyramide, dofetilide, quinidine sulphate.

Anti-bacterial agents: benethamine penicillin, cefrozil, cinoxacin, ciprofloxacin, clarithromycin, clofazimine, cloxacillin, doxycycline, erythromycin, ethionamide, fusidic acid, muciprocin, nalidixic acid, nifuroxazide, nitrofurantoin, oxacillin, rifampicin, sparfloxacin, spiramycin, sulphabenzamide, sulphacetamide, sulphadiazine, sulphadoxine, sulphafurazole, sulphamerazine, sulphamethoxazole, sulphapyridine, telithromycin, tetracycline, trimethoprim, trovafloxacin.

Anti-coagulants: clopidogrel, dicoumarol, dipyridamole, nicoumalone, phenindione, tirofibran.

Anti-depressants: amoxapine, maprotiline, paroxetine, sertraline, trimipramine maleate.

Anti-diabetics: acetohexamide, chlorpropamide, glibenclamide, glimepiride, gliclazide, glipizide, glymepride, pioglitazone, rasiglitazone tolazamide, tolbutamide.

Anti-epileptics: beclamide, carbamazepine, clonazepam, ethotoin, methoin, methsuximide, methylphenobarbitone, phenacemide, phenobarbitone, phenyloin, phensuximide, primidone, sulthiame, tiagabine, valproic acid.

Anti-fungal agents: amphotericin, azithromycin, butoconazole nitrate, clotrimazole, econazole nitrate, fluconazole, griseofulvin, itraconazole, ketoconazole, miconazole, natamycin, nystatin, oxiconazole, sulconazole nitrate, terbinafine, terconazole, tioconazole, tolnaftate, undecenoic acid, voriconazole.

Anti-gout agents: allopurinol, probenecid, sulphin-pyrazone.

Antihistamines: astemizole, cinnarizine, cyclizine, cyproheptadine HCl, dimenhydrinate, fexofenadine, loratadine, meclozine HCl, terfenadine antazoline, azatadine, azelastine, astemazole, bramazine (bromodiphenhydramine), brompheniramine, buclizine, carbinoxamine, cabastin, carebastine, cetirizine, chlorcyclizine, chlorphenamine (chlorpheniramine), chlorphenoxamine, chloropyrilene, cinnarizine, clemastine, clocinizine, cyclizine, cyproheptadine, deptropine, desloratidine, diphenylhydramine, dimenhydrinate, diphenylpyraline, doxylamine, ebastine, embramine, emedastine, epinastine, fexofenadine, flunarizine, halopyramine, histapyrrodine, homochlorcyclizine, hydroxyzine, isothipendyl, levocarbastine, loratadine, mebhydrolin, meclozine, mefenidramium, mepyramine, mequitazine, methdilazine, mizolastine, oloptadine, oxatomide, oxomemazine, phenindamine, pheniramine, phenyltoloxamine, pimethixene, promethazine, promethazine, propiomazine, pyrrobutamine, rupatadine, setastine, talastine, temelastine, terfenadine, thiethylperazine, trimethobenzamide, tripelennamine, triprolidine, tritoqualine

Anti-hypertensive agents: amlodipine, benidipine, candesartan cilexitil, clonidine, darodipine, diazoxide, eprosartan, felodipine, irbesartan, irinotecan, isradipine, losartan, minoxidil, nicardipine HCl, nifedipine, nimodipine, prazosin, raubasine, reserpine, tamsulosin, telmisartan, valsartan.

Anti-malarials: amodiaquine, chloroquine, halofantrine, mefloquine HCl, proguanil HCl, pyrimethamine, quinine sulphate.

Anti-migraine agents: dihydroergotamine mesylate, ergotamine tartrate, methysergide maleate, pizotifen maleate.

Anti-muscarinic agents: atropine, benzhexol HCl, biperiden, hyoscyamine, mepenzolate bromide, tropicamide.

Anti-neoplastic agents and immunosuppressants: aminoglutethimide, amsacrine, anastrazole, azathioprine, bicalutamide, busulphan, chlorambucil, clobetasol, cyclosporine, dacarbazine, estramustine, etoposide, exemestane, gefitinib, letrozole, lomustine, melphalan, mercaptopurine, methotrexate, mitomycin, mitotane, mitozantrone, mycophenolate mofetil, nilutanide, paclitaxel, procarbazine, sirolimus, tacrolimus, tamoxifen, testolactone, toremifine.

Anti-protazoal agents: atovaquone, clioquinol, diiodohydroxyquinoline, diloxanide furoate, dinitolmide, furzolidone, metronidazole, nitrofurazone, ornidazole, timidazole.

Anti-thyroid agents: carbimazole, propylthiouracil.

Anti-Parkinsonian agents: apomorphine, bromocriptine mesylate, tolcapone, selegiline.

Anti-viral agents: adefovir dipovoxil, amprenavir, efavirenz, lopinavir, nelfinavir, penciclovir, ritonavir, saquinavir, tipranavir.

Anxiolytic, sedatives, hypnotics and neuroleptics: alprazolam, amylobarbitone, aripiprazole, barbitone, bromazepam, bromperidol, brotizolam, butobarbitone, carbromal, chlordiazepoxide, chlormethiazole, chlorpromazine, clobazam, clozapine, diazepam, droperidol, eszopaclone, ethinamate, fluanisone, flunitrazepam, fluopromazine, flupenthixol decanoate, fluphenazine decanoate, flurazepam, haloperidol, lorazepam, lormetazepam, medazepam, meprobamate, methaqualone, midazolam, nitrazepam, oxazepam, paroxetine, pentobarbitone, perphenazine pimozide, prochlorperazine, sertindole, sulpiride, temazepam, thioridazine, triazolam, zaleplon, zolpidem, zopiclone.

Beta-blockers: nadolol, pindolol.

Bronchodilators and anti-asthma agents: zafirlukast, zileuton.

Cardiac inotropic agents: digitoxin, digoxin, lanatoside C, medigoxin.

Corticosteroids: beclomethasone, betamethasone, budesonide, clobetasol, clobetasone, cortisone acetate, desonide, desoxymethasone, dexamethasone, fludrocortisone acetate, flunisolide, flucortolone, fluticasone propionate, hydrocortisone, methylprednisolone, mometasone, prednisolone, prednisone, rimexocone, triamcinolone.

Dermatological drugs and retinoids: acitretin, benzoyl peroxide, bexarotene, dapsone, dithranol, isotretinoin, methoxsalen, pimecrolimus, tretinoin.

Diuretics: acetazolamide, amiloride, bendrofluazide, bumetanide, chlorothiazide, chlorthalidone, ethacrynic acid, frusemide, metolazone, polythiazide, spironolactone, triamterene.

Erectile dysfunction: sildenafil, vardenifil, tadalafil.

Gastro-intestinal agents: attapulgite, bisacodyl, cimetidine, cisapride, diphenoxylate, domperidone, dronabinol, droperidol, famotidine, granisetron, lansoprazole, loperamide, magaldrate, mesalazine, nabilone, omeprazole, ondansetron, palonosetron, pizotifen sulphasalazine, tegaserod.

Lipid regulating agents: atorvastatin, bezafibrate, clofibrate, fenofibrate, gemfibrozil, probucol, simvastatin

Muscle relaxants: baclofen, carisoprodol, chlorzoxone, tizanidine.

Nitrates and other anti-anginal agents: amyl nitrate, glyceryl trinitrate, isosorbide mononitrate, pentaerythritol tetranitrate.

Nutritional agents: betacarotene, essential fatty acids, vitamin A, vitamin B₂, vitamin B₁₂, vitamin D, vitamin E, vitamin K (menadione, phytomenadione).

Opioid analgesics: codeine, dextropropyoxyphene, diamorphine, fentanyl, meptazinol, morphine, pentazocine.

Sex hormones: clomiphene citrate, danazol, ethinyl estradiol, finasteride, medroxyprogesterone acetate, megestrol, mestranol, methyltestosterone, norethisterone, norgestrel, estradiol, conjugated oestrogens, progesterone, raloxifene, stanozolol, stibestrol, testosterone, tibolone.

Stimulants and anoretics: dexamphetamine, dexfenfluramine, mazindol, sibutramine.

The viral vector may be a retrovirus (such as Moloney murine leukaemia virus), a lentivirus, an adenovirus, an adeno-associated virus (AAV) or a nanoengineered substance such as Ormosil.

Other pharmaceutically acceptable salts, isomers, esters and derivatives thereof and bases may be substituted for these drugs. Mixtures of lipophilic drugs may be used where therapeutically effective.

The pharmaceutical composition of the present invention is preferably presented in a unit dosage form. Each unit dosage may comprise from 0.0025 mg to 500 mg, and in particular from 1 mg to 100 mg, of the pharmaceutically active agent. It will be understood that the preferred unit dosage will depend on the particular pharmaceutically active agent used, or the particular combination of pharmaceutically active agents used, and the method of application of the dosage.

Either or both the hydrophilic and/or hydrophobic phase of the polyaphron dispersions and/or the polymer matrix of the present invention may include antioxidants, preservatives, pH modifiers, sequestering agents and other lipophilic or hydrophilic additives known to improve the stability and longevity of oil-in-water dispersions and polymer solutions, preferably aqueous based polymer systems. Such additives include for example, but are not limited to, tert-butylhydroquinone, butylated hydroxylanisole, tocopherols and propyl gallate (antoxidants), methyl, propyl and butyl parabens, phenoxyethanol (preservatives), ethylene diamine tetra acetic acid—EDTA (sequestering agent), citric acid, sodium hydroxide (pH modifiers) and others known to those skilled in the art.

A variety of flavouring agents may also be added to the pharmaceutical compositions. Any suitable amount and type of artificial and/or natural flavouring agents can be used in any sensorially acceptable fashion. For example, the flavour can constitute from 0.05% to 20% by weight of the total weight of the pharmaceutical composition, preferably 0.1% to 5%. The flavouring agents can include, for example, essential oils, synthetic flavours or mixtures, including but not limited to, oils delivered from plants and fruits such as citrus oils, fruit essences, peppermint oil, spearmint oil, other mint oils, clove oils, oil of wintergreen, anise and the like, flavouring substances with germ killing properties such as menthol, eucalyptol, thymol, like flavouring agents or combinations thereof.

Colouring agents may be included in the pharmaceutical composition. These may include, for example, natural food colours and dyes suitable for drug applications, and may be present in amounts from 0.01% to 1.5% by weight of the total pharmaceutical composition.

In one embodiment of the present invention the pharmaceutical composition comprises a transparent polymer matrix and coloured (non-transparent) aphrons. Similar such embodiments may be designed with the aim of making the pharmaceutical composition particularly attractive to the end user.

The pharmaceutical composition of the present invention may comprise an opacifer, or a mixture of opacifiers/fillers. The opacifier may be added in order to obtain an opaque moulded body or in order to protect light sensitive pharmaceutically active agents dispersed within the composition. Opacifiers may be present in an amount of from 0.01% to 15% by weight, preferably 0.1% to 10% by weight of the total weight of the pharmaceutical composition. Examples of suitable opacifiers include titanium dioxide, calcium carbonate, iron oxide, glycol stearate, kaolin and bentonite clays. Preferably the opacifier is titanium dioxide.

In one embodiment of the present invention the pharmaceutical composition comprises from 10 to 20% by weight of polymer, from 40 to 70% by weight of discontinuous phase of polyaphron dispersion and from 0.01 to 10% by weight of at least one pharmaceutically active agent, the balance being water or other suitable solvent.

In another embodiment of the present invention the pharmaceutical composition comprises from 10 to 20% by weight of polymer, from 40 to 70% by weight of discontinuous phase of polyaphron dispersion and from 0.01 to 5% by weight of at least one pharmaceutically active agent, the balance being water or other suitable solvent.

In a further embodiment of the present invention the pharmaceutical composition comprises from 20 to 35% by weight of polymer, from 40 to 70% by weight of discontinuous phase of polyaphron dispersion and from 0.1 to 2% by weight of at least one pharmaceutically active agent, the balance being water or other suitable solvent.

In another embodiment of the present invention the pharmaceutical composition comprises from 20 to 35% by weight of polymer, from 40 to 60% by weight of discontinuous phase of polyaphron dispersion and from 0.1 to 2% by weight of at least one pharmaceutically active agent, the balance being water or other suitable solvent.

In a preferred embodiment of the present invention the polymer is gelatine, the polyaphron dispersion comprises oil and water, and at least one pharmaceutically active agent is a poorly water soluble drug. Preferably at least one pharmaceutically active agent is dispersed and/or dissolved in the discontinuous pharmaceutical oil phase of the polyaphron dispersions. Preferably in this embodiment the pharmaceutical composition is moulded into the form of a capsule, pessary, suppository, or film.

In a further aspect of the present invention there is provided a pharmaceutical composition as described herein for use in a method of treatment of the human or animal body by therapy.

In another aspect of the present invention there is provided the use of the pharmaceutical composition as described herein for the manufacture of a medicament for treating, allergic reactions, alopecia, anaemia, angina, anxiety disorders, arrhythmia, arthritis, asthma, bites and stings, bone diseases, cancer, coughs, dementia, depression, diabetes, drug addiction, eczema, elevated cholesterol, epilepsy, extrapyramidal disorders, fever and hyperthermia, gastrointestinal disorders, gout, heart failure, hormonal disorders, hyperlipidaemia, hypertension, hypochondria, hypothalamic and pituitary disorders, infections (bacterial, ectoparastic, fungal, helminthic, protozoal, viral), inflammation, insomnia and parasomnias, protozoal infections, kidney disorders, labour induction, liver disorders, local and systemic pain, malaria, migraine, muscular dystrophies, myasthenia, nasal congestion, nutritional deficiencies, parathyroid disorders, Parkinson's disease, poisoning, premature labour, provide sedation, psychoses, respiratory disorders, rheumatism, salt retention, shock, skin disorders (acne, dermatitis, keratinisation disorders, psoriasis, ulcers, warts), thromboembolic disorders, tapeworm infection, thyroid imabalnce, tuberculosis, urological disorders, water retention, local or systemic pain or xenograft rejection.

In another aspect of the present invention there is provided a pharmaceutical composition as described herein for use in treating allergic reactions, alopecia, anaemia, angina, anxiety disorders, arrhythmia, arthritis, asthma, bites and stings, bone diseases, cancer, coughs, dementia, depression, diabetes, drug addiction, eczema, elevated cholesterol, epilepsy, extrapyramidal disorders, fever and hyperthermia, gastrointestinal disorders, gout, heart failure, hormonal disorders, hyperlipidaemia, hypertension, hypochondria, hypothalamic and pituitary disorders, infections (bacterial, ectoparastic, fungal, helminthic, protozoal, viral), inflammation, insomnia and parasomnias, protozoal infections, kidney disorders, labour induction, liver disorders, local and systemic pain, malaria, migraine, muscular dystrophies, myasthemia, nasal congestion, nutritional deficiencies, parathyroid disorders, Parkinson's disease, poisoning, premature labour, provide sedation, psychoses, respiratory disorders, rheumatism, salt retention, shock, skin disorders (acne, dermatitis, keratinisation disorders, psoriasis, ulcers, warts), thromboembolic disorders, tapeworm infection, thyroid imabalnce, tuberculosis, urological disorders, water retention, local or systemic pain or xenograft rejection.

In another aspect of the present invention there is provided a method of making the pharmaceutical composition as defined herein comprising mixing a polyaphron dispersion with a polymer to form a polymer matrix having a polyaphron dispersion and at least one pharmaceutically active agent dispersed therein, and moulding the mixture into a desired shape.

In one embodiment, the method of making the pharmaceutical composition according to the present invention comprises:

(i) providing a polymer solution comprising a polymer and a solvent, and optionally at least one pharmaceutically active agent;

(ii) mixing the polymer solution with at least one polyaphron dispersion, which optionally comprises at least one pharmaceutically active agent, to form a mixture;

(iii) moulding said mixture to provide a pharmaceutical composition comprising a moulded body of a polymer matrix.

The pharmaceutically active agent may be contained with the polyaphron dispersion. Alternatively, or additionally the pharmaceutically active agent may be added to or provided in the polymer solution.

In a further embodiment of the method of making the pharmaceutical composition of the present invention the mixture provided in step (iii) as described above is formed into a moulded body by, optionally transferring said mixture into a mould, and by changing the temperature of the mixture and/or by the addition of a cross-linking agent to the mixture and/or by the activation or a cross-linking agent.

In a further embodiment of the method of the present invention the polymer solution is in the form of a sol, which is capable of undergoing a sol-gel transition to form a gel which is capable of being moulded into a desired shape.

In a further aspect of the present invention there is provided a pharmaceutical composition as produced by the method as described herein.

In one embodiment of the present invention the mixture may be moulded into the desired shape by, for example, casting, centrifugal casting, extrusion, injection moulding, blow moulding or dipping. Such methods are well known in the art.

In a preferred method of moulding, the liquid dispersion of polyaphron dispersion in the polymer in sol or uncrosslinked form is poured or injected into moulds the size and shape of gelatine capsules, lozenges, tablets, or pessaries, and the liquid contents of the mould are formed into a semi-solid or solid form by change of temperature or crosslinking with a suitable crosslinking agent. The moulded products are subsequently removed from the moulds (by the use, for example, of split moulds) and are then ready for transferring into suitable packaging prior to sale or use by the intended patient.

The pharmaceutically active agent(s) may be dispersed and/or dissolved in the continuous and/or discontinuous phase prior to forming the aphron dispersions.

In one embodiment of the present invention the pharmaceutically active drug is dissolved in a suitable liquid lipid solvent(s). The aphrons droplets are formed using the solution described above as the discontinuous phase by methods known in the art. The polyaphron dispersions are then mixed with a suitable polymer in sol form (for example gelatine solution which has been heated to 40° C. or a solution of the sodium salt of an alginate). A plasticizer such as glycerine, sorbitol or a suitable glycol (for example, propylene glycol, or polyethylene glycols) may be added to the mixture. The mixture is then poured into a mould of the desired shape, or formed into a cast film and a sol/gel transition in brought about (for example, by cooling the mixture to room temperature) or a solidification process is brought about by crosslinking by the use of (for example) suitable metal ions such as Ca²⁺.

One advantage of the present invention is that wherein at least one pharmaceutically active agent is dissolved and/or dispersed in the oil discontinuous phase of the aphrons the pharmaceutically active agent has a better chance of being absorbed through the gut wall or epithelial lining of the mouth than with a simple solution of pharmaceutically active agent in oil. At the same time, each droplet is water-dispersible and therefore capable of existing as a discrete and separate entity with minimal risk of coalescence to produce larger droplets, so increasing the overall surface area available through which the drug molecules can be absorbed in order to reach the systematic circulation.

The present inventors have also observed that the pharmaceutical compositions as described herein are capable of floating on water whilst dissolving and/or dispersing. Thus, the pharmaceutical compositions may be suitable for use as floating drug delivery systems. Such systems are, for example, useful in the gastrointestinal tract and in particular, in the stomach, where the floating system is prevented from being flushed into the small intestine by floatation on the stomach contents. The floating drug delivery system as described herein has the advantage that the drug may be released over an extended period of time, as the pharmaceutical composition gradually dissolves and/or disintegrates. Thus the pharmaceutical composition may be regarded as a gastroretentive dosage form (GRDF). Other GRDF exist in the prior art, but these are invariably complex and difficult to manufacture. In contrast to this, the present invention has the advantage that it is low cost and simple to manufacture, whilst being effective.

In one embodiment of the present invention there is provided a oral drug delivery system comprising the pharmaceutical composition as described herein. The oral drug delivery system may be designed for swallowing, chewing, as a floating drug delivery system, or as a lozenge.

In another embodiment of the present invention there is provided a wound dressing, a pessary, or a suppository comprising the pharmaceutical composition as described herein.

The following Examples further illustrate the present invention.

### EXAMPLE 1

### A Pharmaceutical Composition was Prepared in the Following Way

Two constituents were prepared for the composite. The polymer solution contained 40% w/w porcine gelatine and 5% w/w sorbitol (plasticiser) in water. The polyaphron dispersion phase contained a lipid phase (90% w/w of total polyaphron dispersion) consisting of 5.6% w/w cyclosporine, 41.5% w/w peanut oil, 41.5% Maisine 35-1, 1% w/w Span 20, 1% oleic acid, and an aqueous phase consisting of 10% w/w Poloxamer 188 in water.

The polymer solution was warmed to 50° C. and the polymer solution and the polyaphron dispersion were mixed such that the polymer solution contributed 33.8% w/w of the total formulation.

### The Final Formulation was:

| | |
|---|---|
| Gelatine | 13.62% |
| Cyclosporine | 3.33% |
| Peanut oil | 27.68% |
| Maisine 35-1 | 27.68% |
| Span 20 | 0.60% |
| Oleic acid | 0.60% |
| Sorbitol | 1.70% |
| Water | 24.79% |

A 750 mg mass of this formulation contains 25 mg cyclosporine.

The resulting formulation, whilst warm (above approximately 40° C.) was pourable and easily mouldable. Once set, the moulded formulation did not leak oil, and was able to remain pliable (i.e. did not harden) when stored at room temperature in a sealed container.

### EXAMPLE 2

Two constituents were prepared for the composite. The polymer solution contained 40% w/w porcine gelatine and 5% w/w glycerol (plasticiser) in water. The polyaphron dispersion phase contained a lipid phase (90% w/w of total polyaphron dispersion) consisting of 8.0% w/w halofantrine base, 82.0% w/w capric and caprylic triglycerides (Miglyol-812), and an aqueous phase consisting of 15% w/w Cremophor RH40 (hydrogenated castor oil ethylene oxide 40) in water.

The polymer solution was warmed to 50° C. and the polyaphron dispersion and the polymer solution were mixed such that the polymer solution phase contributed 37.5% w/w of the total formulation.

### The Final Formulation was:

| | |
|---|---|
| Gelatine | 15.00% |
| Halofantrine | 5.00% |
| Miglyol-812 | 51.25% |
| Cremophor RH40 | 0.94% |
| glycerol | 1.88% |
| Water | 25.93% |

A 1000 mg mass of this formulation contains 50 mg halofantrine.

The resulting formulation, whilst warm (above approximately 40° C.) was pourable and easily mouldable into suitable forms. Once set, the moulded formulation did not leak oil, and was able to remain pliable when stored at room temperature in a sealed container.

### EXAMPLE 3

Two constituents were prepared for the composite. The polymer solution contained 32.6% w/w porcine gelatine and 8.6% w/w powdered, food grade titanium dioxide (opacifier) in water. The polyaphron dispersion phase contained soyabean oil phase and an aqueous phase consisting of poloxamer 188 and span 80 (emulsifiers) in water.

### The Polyaphron Dispersion Formulation was:

| | |
|---|---|
| Soyabean oil | 89.00% |
| Span 80 | 1.00% |
| Water | 9.90% |
| Poloxamer 188 | 0.10% |

### The Final Formulation was:

| | |
|---|---|
| Gelatine | 22.90% |
| Water | 44.60% |
| Titanium Dioxide | 6.10% |
| Soyabean Oil | 25.82% |
| Span 80 | 0.29% |
| Poloxamer 188 | 0.29% |

The polymer solution was warmed to 50° C. and the polymer solution and the polyaphron dispersion were mixed such as to give the above final formulation.

### EXAMPLE 4

Two constituents were prepared for the composite. The polymer solution contained carrageenan gum and the polyaphron phase contained sunflower oil as follows:

### The Polyaphron Dispersion Formulation was:

| | |
|---|---|
| Sunflower oil | 90.00% |
| Cremophor RH40 | 0.90% |
| Crill 4 | 0.90% |
| Water | 8.20% |

### The Final Formulation was:

| | |
|---|---|
| k-carragenan | 2.80% |
| Polyaphron Dispersion | 30.00% |
| Water | 67.20% |

The polymer solution was heated to 90° C. and the polymer solution and the polyaphron dispersion were mixed and poured into a mould and allowed to set by sol-gel transition. The resulting product was a firm gel, which floated in water.

### EXAMPLE 5

Two constituents were prepared for the composite. The polymer solution contained carrageenan gum and sodium alginate and the polyaphron phase contained sunflower oil as follows:

### The Polyaphron Dispersion Formulation was:

| | |
|---|---|
| Sunflower oil | 90.00% |
| Cremophor RH40 | 0.90% |
| Crill 4 | 0.90% |
| Water | 8.20% |

### The Final Formulation was:

| | |
|---|---|
| k-carrageenan | 4.00% |
| Kelton LVCR | 1.00% |
| Polyaphron Dispersion | 30.00% |
| Water | 65.00% |

The polymer solution was heated to 90° C. and the polymer solution and the polyaphron dispersion) were mixed and poured into a mould and allowed to set by sol-gel transition. The resulting product was placed in 5% w/w calcium chloride solution for 5 minutes. This resulted in a very firm gel, which sinks in water.

### EXAMPLE 6

Two constituents were prepared for the composite. The polymer solution contained Poly(vinyl pyrrolidone) and Gelatine. The polyaphron phase was the sunflower oil dispersion used in Example 5.

| | |
|---|---|
| Poly(vinyl pyrrolidone) | 10.00% |
| Gelatine | 25.00% |
| Polyaphron Dispersion | 8.75%% |
| Water | 56.25 |

The polymer solution was heated to 90° C. and the polymer solution and the polyaphron dispersion were mixed and poured into a mould and allowed to set by sol-gel transition. The resulting product was placed in 5% w/w calcium chloride solution for 5 minutes. This resulted in a very firm gel, which sinks in water.

In all of the above examples, the (hot) compositions were poured into a suppository mould and allowed to cool. The resulting moulded forms of the invention were gel-like and suppository shaped.

### Footnote to the Examples

| Trade Name | Supplier | INCI Name |
|---|---|---|
| Poloxamer 188 | BASF | Poloxamer 188 |
| Span 80 | Surfachem UK | Sorbitan Oleate |
| Cremophor RH40 | BASF | PEG-40 Hydrogenated Castor Oil |
| Miglyol-812 | S Black | Caprylic/Capric Triglyceride |
| Maisine 35-1 | Gattefossé | Propylene Glycol Caprylate. |
| Kelton LVCR | ThewArnott | Algin |
| Crill 4 | Croda | Sorbitan Oleate |
| k-carrageenan | ISP | Carrageenan |

1. A pharmaceutical composition comprising a moulded body of a polymer matrix, said polymer matrix comprising at least one polyaphron dispersion and at least one pharmaceutically active agent dispersed therein.

2. The pharmaceutical composition of claim 1 wherein the composition comprises at least 10% by weight of solvent based on the total weight of total composition.

3. The pharmaceutical composition of claim 2 wherein the composition comprises at least 20% by weight of solvent based on the total weight of total composition.

4. The pharmaceutical composition of claim 2 wherein the solvent is selected from one or more of an aliphatic alcohol, polyethylene glycol, propylene glycol, glycerol, and water.

5. The pharmaceutical composition of claim 1 wherein pharmaceutical composition comprises at least 55% by weight of at least one pharmaceutically acceptable oil in the polyaphron dispersion based on the total weight of the pharmaceutical composition.

6. The pharmaceutical composition of claim 1 comprising from 0.0001 to 40% by weight of at least one pharmaceutically active agent based on the total weight of the pharmaceutical composition.

7. The composition of claim 1 wherein the moulded body of the polymer matrix is formed by sol-gel transition of a polymer comprising at least one polyaphron dispersion and at least one pharmaceutically active agent dispersed therein as a result of cross-linking the polymer or as a result of a temperature change.

8. The pharmaceutical composition of claim 1 wherein the polymer matrix comprises cross-linked polymers.

9. The pharmaceutical composition of claim 1 wherein at least one of said pharmaceutically active agents is at least partially dispersed and/or dissolved in the discontinuous phase of the polyaphron dispersion.

10. The pharmaceutical composition of claim 1 wherein at least one of said pharmaceutically active agents is at least partially dispersed and/or dissolved in the polymer matrix.

11. The pharmaceutical composition of claim 1 wherein the polymer is gelatine, carrageenan, or an alginate.

12. The pharmaceutical composition of claim 1 wherein the moulded polymer matrix is in the form of a semi-solid colloidal gel.

13. The pharmaceutical composition of claim 1 in the form of a capsule, suppository, pessary, depot, a lozenge or a film.

14. The pharmaceutical composition of claim 1 which is in unit dosage form.

15. The pharmaceutical composition of claim 1 comprising a pharmaceutically active agent selected from an analgesic or anti-inflammatory agent, an anthelmintic, an anti-arrhythmic agent, an anti-coagulant, an anti-depressant, an anti-diabetic, an anti-epileptic, an anti-fungal agent, an anti-gout agent, an anti-hypertension agent, an anti-malarial, an anti-migraine agent, an antimuscarinic agent, an anti-neoplastic agent, an antiprotozoal agent, an anti-thyroid agent, an anxiolytic, sedative, hypnotic or neuroleptic agent, a corticosteroid, a diuretic, an anti-Parkinsonian agent, a gastro-intestinal agent, a histamine Hi-receptor antagonist, a lipid regulating agent, an anti-anginal agent, a thyroid agent, a nutritional agent, an antipyretic agent, an antibacterial agent, an immunosuppressant, an antiviral agent, a hypothalamic or a pituitary hormone, a sex hormone, a prostaglandin, a vaccine, a cough suppressant, a local anaesthetics, an immuno-globulin, an antisera, an opioid analgesic, a stimulant, a viral vector for gene therapy, or a therapeutic mixture thereof.

16. A method of making the pharmaceutical composition as defined in claim 1 comprising mixing a polyaphron dispersion with a polymer to form a polymer matrix having a polyaphron dispersion and at least one pharmaceutically active agent dispersed therein, and moulding the mixture into a desired shape.

17. The method of making the pharmaceutical composition according to claim 16 comprising:
(i) providing a polymer solution comprising a polymer and a solvent, and optionally at least one pharmaceutically active agent;
(ii) mixing the polymer solution and at least one polyaphron dispersion, which optionally comprises at least one pharmaceutically active agent, to form a mixture;
(iii) moulding said mixture to provide a pharmaceutical composition comprising a moulded body of a polymer matrix.

18. The method of making the pharmaceutical composition according to claim 17 wherein the mixture is formed into a moulded body by, optionally transferring said mixture into a mould, and by changing the temperature of the mixture and/or by the addition of a cross-linking agent to the mixture.

19. The method of claim 17 wherein the polymer solution is in the form of a sol, which is capable of undergoing a sol-gel transition to form a gel which is capable of being moulded into a desired shape.

20. The pharmaceutical composition as produced by the method as defined in claim 16.

21. The pharmaceutical composition of claim 1 further comprising an outer coating.

22. The pharmaceutical composition as defined in claim 1 for use in a method of treatment of the human or animal body by therapy.

23. The pharmaceutical composition as defined claim 1 for use in treating allergic reactions, alopecia, anaemia, angina, anxiety disorders, arrhythmia, arthritis, asthma, bites and stings, bone diseases, cancer, coughs, dementia, depression, diabetes, drug addiction, eczema, elevated cholesterol, epilepsy, extrapyramidal disorders, fever and hyperthermia, gastrointestinal disorders, gout, heart failure, hormonal disorders, hyperlipidaemia, hypertension, hypochondria, hypothalamic and pituitary disorders, infections (bacterial, ectoparastic, fungal, helminthic, protozoal, viral), inflammation, insomnia and parasomnias, protozoal infections, kidney disorders, labour induction, liver disorders, local and systemic pain, malaria, migraine, muscular dystrophies, myasthemia, nasal congestion, nutritional deficiencies, parathyroid disorders, Parkinson's disease, poisoning, premature labour, provide sedation, psychoses, respiratory disorders, rheumatism, salt retention, shock, skin disorders (acne, dermatitis, keratinisation disorders, psoriasis, ulcers, warts), thromboembolic disorders, tapeworm infection, thyroid imbalance, tuberculosis, urological disorders, water retention, local or systemic pain or xenograft rejection.

24. Use of the pharmaceutical composition as defined in claim 1 for the manufacture of a medicament for allergic reactions, alopecia, anaemia, angina, anxiety disorders, arrhythmia, arthritis, asthma, bites and stings, bone diseases, cancer, coughs, dementia, depression, diabetes, drug addiction, eczema, elevated cholesterol, epilepsy, extrapyramidal disorders, fever and hyperthermia, gastrointestinal disorders, gout, heart failure, hormonal disorders, hyperlipidaemia, hypertension, hypochondria, hypothalamic and pituitary disorders, infections (bacterial, ectoparastic, fungal, helminthic, protozoal, viral), inflammation, insomnia and parasomnias, protozoal infections, kidney disorders, labour induction, liver disorders, local and systemic pain, malaria, migraine, muscular dystrophies, myasthemia, nasal congestion, nutritional deficiencies, parathyroid disorders, Parkinson's disease, poisoning, premature labour, provide sedation, psychoses, respiratory disorders, rheumatism, salt retention, shock, skin disorders (acne, dermatitis, keratinisation disorders, psoriasis, ulcers, warts), thromboembolic disorders, tapeworm infection, thyroid imabaince, tuberculosis, urological disorders, water retention, local or systemic pain or xenograft rejection.
